(19) United States
(12) Patent Application Publication
(10) Pub. No.: US 2023/0044507 A1
(43) Pub. Date: Feb. 9, 2023

(54) PULP CAVITY DISTANCE MEASUREMENT SYSTEM AND METHOD

(71) Applicant: MEDIT CORP., Seoul (KR)

(72) Inventor: Myoung Woo SONG, Seoul (KR)

(73) Assignee: MEDIT CORP., Seoul (KR)

(21) Appl. No.: 17/974,485

(22) Filed: Oct. 26, 2022

Related U.S. Application Data
(63) Continuation of application No. PCT/KR2021/005242, filed on Apr. 26, 2021.

(30) Foreign Application Priority Data
Apr. 28, 2020 (KR) 10-2020-0051644
Apr. 26, 2021 (KR) 10-2021-0053449

(51) Int. Cl. A61B 5/107; A61B 1/24; A61B 1/00; A61B 5/00; A61C 9/00

(52) U.S. Cl. CPC A61B 5/1076; A61B 1/24; A61B 1/00172; A61B 5/0088; A61B 5/743; A61C 9/0053

(57) ABSTRACT

A pulp cavity distance measurement system according to the present invention comprises: a first scan unit which acquires surface data; and a second scan unit which acquires volume data that is scan information different from the surface data, wherein the surface data and volume data are delivered via a database unit included in a control unit, and are merged into a single piece of data by a data merging unit. The shortest distance (pulp cavity distance) from the surface of the enamel of a tooth to the surface of the pulp cavity is calculated from the merged data, and the pulp cavity distance may be visually displayed by a distance stage display unit by using the calculated distance information (data). Here, the visual display may be expressed using colors or patterns having specific markings, and have a plurality of patterns so that the distance is displayed so as to be divided in stages. Accordingly, the system is advantageous in that a therapist can minimize tooth preparation in a part in which the distance to the pulp cavity is short, and reduce the discomfort of a patient due to vibrations caused by tooth preparation.

### TECHNICAL FIELD

The present disclosure relates to a pulp cavity distance measurement system and method.

### BACKGROUND ART

In dental care, a process of deleting a decayed part of a tooth by using a grinder for dental care has been frequently performed. In this case, if patient's nerves are formed adjacent to a location where tooth preparation is in progress, vibrations that occur in the process of tooth preparation may be transferred to the nerves. If such vibrations are transferred to the nerves, the nerve bundle trembles, and thus the patient may feel hypersensitivity or toothache to cause discomfort to the patient.

Meanwhile, the tooth preparation may be performed to simply remove the decayed (damaged) part of the tooth, or may sometimes be performed for the purpose of properly shaping the appearance of the tooth to apply a prosthetic structure, such as implant or crown treatment, into the patient's oral cavity. Except a case of removing the tooth completely by extracting the tooth that is located in the gingiva, the tooth to be treated should be molded so that the tooth is combined with the prosthetic treatment that a therapist (typically corresponding to a dentist who performs dental care and treatment) intends to apply to the tooth with a gap between the tooth and the prosthetic treatment minimized.

### DISCLOSURE

### Technical Problem

The present disclosure provides a pulp cavity distance measurement system, which marks the distance from an enamel surface to a pulp cavity surface.

Further, the present disclosure provides a pulp cavity distance measurement method, which has steps of acquiring data of a patient's tooth and a tooth model through a pulp cavity distance measurement system and calculating a distance from an enamel surface of the tooth to a pulp cavity surface accordingly.

The technical problems of the present disclosure are not limited to the above-described technical problems, and other unmentioned technical problems may be clearly understood by those skilled in the art from the following descriptions.

### Technical Solution

A pulp cavity distance measurement system according to the present disclosure may include: a database unit configured to acquire surface data of a tooth or a tooth model and volume data of the tooth; and a calculation unit configured to calculate a distance between corresponding parts of a 3D surface model implemented from the surface data and a 3D volume model implemented from the volume data after aligning the 3D surface model and the 3D volume model.

Further, the system may further include a first scan unit configured to acquire and transfer the surface data to the database unit.

Further, the system may further include a second scan unit configured to acquire and transfer the volume data to the database unit.

Further, the 3D volume model may include data from a surface of the tooth to a pulp cavity surface inside the tooth.

Further, the calculation unit may be configured to calculate a distance after aligning between a tooth surface of the 3D surface model and a tooth surface of the 3D volume model.

Further, the calculation unit may be configured to calculate a distance from a tooth surface of the 3D surface model to a pulp cavity surface of the 3D volume model.

Further, the distance may be the shortest distance from a measurement point of the 3D surface model to the pulp cavity surface of the 3D volume model.

Further, the system may further include a distance stage display unit configured to visually display the distance.

Further, the distance stage display unit may be configured to separately display a plurality of patterns in accordance with a size of the distance.

Further, the plurality of patterns may be separately displayed with different colors.

A pulp cavity distance measurement method according to the present disclosure may include: a data acquisition step of acquiring surface data of a tooth or a tooth model and volume data of the tooth; a data merging step of merging a 3D surface model implemented from the surface data with a 3D volume model implemented from the volume data; and a distance calculating step of calculating a distance between corresponding parts of the 3D surface model and the 3D volume model after aligning the 3D surface model and the 3D volume model.

Further, the distance calculating step may calculate a distance after aligning between a tooth surface of the 3D surface model and a tooth surface of the 3D volume model.

Further, the distance calculating step may calculate a distance from a tooth surface of the 3D surface model to a pulp cavity surface of the 3D volume model.

Further, the distance calculated in the distance calculating step may be the shortest distance from a measurement point of the 3D surface model to the pulp cavity surface of the 3D volume model.

Further, the method may further include a pattern giving step of appearing in a pattern form on a distance stage display unit in order to visually display the distance calculated in the distance calculating step.

Further, a plurality of patterns may be formed in accordance with the distance calculated in the distance calculating step, and may be separately displayed with different colors.

### Advantageous Effects

By using the pulp cavity distance measurement system and method according to the present disclosure, there is an advantage that the therapist can perform the tooth preparation to the point where the patient's discomfort is not caused in the process in which the therapist performs the preparation of the patient's tooth.

Further, since information on the enamel shape that is outwardly shown and information on the pulp cavity surface that is internally shown are used together through data merge between the surface data acquired from a plurality of scan units and volume data, the tomographic distance according to the analysis of CT data is measured and displayed on the screen, and thus the therapist can visually recognize the part to be noted with ease during the tooth preparation.

### DESCRIPTION OF DRAWINGS

FIG. 1 is a diagram illustrating the overall structure of a gingiva and a tooth that constitute an oral cavity.

FIG. 2 is a diagram schematically illustrating the configuration of a pulp cavity distance measurement system according to the present disclosure.

FIG. 3 is a diagram schematically illustrating information inside a tooth that is displayed by using a pulp cavity distance measurement system according to the present disclosure.

FIG. 4 is a schematic flowchart illustrating a pulp cavity distance measurement method according to the present disclosure.

### EXPLANATION OF SYMBOLS

10: gingiva

12: pulp cavity

14: pulp cavity surface (boundary surface)

20: tooth

22: enamel

24: enamel surface

d: distance

L1: first pattern

L2: second pattern

L3: third pattern

100: first scan unit

200: second scan unit

300: controller

310: database unit

320: data merging unit

330: calculation unit

400: distance stage display unit

510: data acquiring step

S20: data merging step

S30: distance calculating step

S40: pattern giving step

### MODE FOR INVENTION

Hereinafter, some embodiments of the present disclosure will be described in detail with reference to the exemplary drawings. In adding reference numerals to constituent elements in the drawings, it is to be noted that the same constituent elements have the same reference numerals as much as possible even if they are represented in different drawings. Further, in explaining embodiments of the present disclosure, the detailed explanation of related known configurations or functions will be omitted if it is determined that the detailed explanation interferes with understanding of the embodiments of the present disclosure.

The terms, such as “first, second, A, B, (a), and (b)”, may be used to describe constituent elements of embodiments of the present disclosure. The terms are only for the purpose of discriminating one constituent element from another constituent element, but the nature, the turn, or the order of the corresponding constituent elements is not limited by the terms. Further, unless otherwise defined, all terms (including technical and scientific terms) used herein have the same meanings as those commonly understood by those ordinary skilled in the art to which the present disclosure belongs. The terms that are defined in a generally used dictionary should be interpreted as meanings that match with the meanings of the terms from the context of the related technology, and they are not interpreted as an ideal or excessively formal meaning unless clearly defined in the present disclosure.

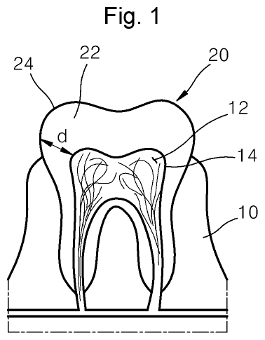

FIG. 1 is a diagram illustrating the overall structure of a gingiva and a tooth that constitute an oral cavity.

Referring to FIG. 1, a tooth 20 is typically formed to get stuck in the gingiva 10 and to surround an outer periphery of the tooth 20, and is supported so that the location and the direction of the tooth 20 formed inside the oral cavity are not changed. In this case, the part of the tooth 20 being supported by the gingiva 10 is referred to as a root, and a part being exposed to an outside of the gingiva to perform chewing is referred to as a crown.

Further, an outer surface of the crown exposed to the oral cavity is formed of an enamel 22. The enamel 22 is the hardest part of the crown surface, and serves to protect the internal structure of the tooth 20 against the chewing pressure in accordance with the chewing action and an acid or a temperature change that causes a decayed tooth. Since the enamel 22 is formed of enamel bars being bent along its whole shape, and the enamel bars are formed of hydroxyapatite crystal, a fine space or gap having no crystal exists between the enamel bars. Due to such structural features, the enamel 22 has various densities and hardness, and if fine particles penetrate inside the enamel 22, dental caries may occur.

Dentin is located under the enamel 22. The dentin has lower hardness than that of the enamel 22, and has elasticity. As compared with the enamel 22 having high hardness, the dentin having high elasticity may serve to prevent the fracture of the enamel 22 by absorbing partial impact when a chewing force (or chewing pressure) in accordance with the chewing action is applied to the tooth 20. Further, since the dentin surrounds the pulp cavity 12, it may also serve to protect the pulp cavity 12 from the above-described external impact.

Meanwhile, inside the tooth, the pulp cavity 12 is formed to serve as a path (space) through which blood vessels and nerve bundles pass. The pulp cavity 12 forms a space having an alphabet “M” shape in cross section, and may have a similar shape to the shape of the exterior of the crown part. The blood vessels and nerve bundles pass through the inside of the pulp cavity 12, and are connected to nerves and blood vessels in the surrounding bone through an apical foramen. In this case, the blood vessels existing inside the pulp cavity 12 provide nutrients to the dentin, and the nerves make the dentin feel the sensation. When unnecessary stimulation occurs on the tooth 20, the nerves sense this, and protect the tooth 20 through an ache reaction (sensitive tooth or toothache).

Meanwhile, among the unnecessary stimulations occurring on the tooth 20, vibrations occurring in accordance with the above-described tooth preparation may be included, and an excessive deletion of the enamel 22 may occur due to the therapist's carelessness or cognitive deficit of the tooth state. This may disturb the matching between the prosthetic treatment and the tooth, and it may be easy for foreign matters to penetrate between the prosthetic treatment and the tooth to rather deteriorate the tooth state. Further, due to the excessive deletion of the enamel 22, the dentin may be damaged in the tooth preparation, and the foreign matters may easily penetrate into the dentin and the pulp cavity 12. Accordingly, for good oral health of the patient, there is a need for a means or a method for a therapist to quickly and easily recognize the distance (hereinafter, distance d) from the enamel surface 24 to the pulp cavity surface 14.

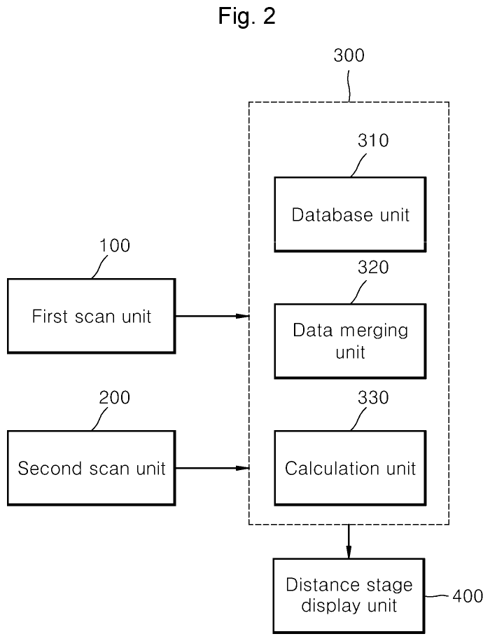

FIG. 2 is a diagram schematically illustrating the configuration of a pulp cavity distance measurement system according to the present disclosure.

Referring to FIG. 2, a pulp cavity distance measurement system according to the present disclosure may include a database unit 310 configured to acquire surface data of a patient's tooth or a tooth model and volume data of the tooth, and a calculation unit 330 configured to calculate a distance between corresponding parts of a 3D surface model implemented from the surface data and a 3D volume model implemented from the volume data after aligning the 3D surface model and the 3D volume model. In this case, the surface data to implement the 3D surface model may be acquired from a firs scan unit 100, and the volume data to implement the 3D volume model may be acquired from a second scan unit 200.

The first scan unit 100 may be a handheld type oral scanner that can scan the oral cavity of a patient through an opening formed at one end part thereof in a manner that one part thereof enters into or is drawn out of the patient's oral cavity. The first scan unit 100 can scan the patient's tooth or the tooth model. The first scan unit 100 formed in the shape of an oral scanner may include at least one camera provided therein and an imaging sensor connected to the camera in a telecommunication manner, and may generate surface data by using light incident through a lens of the camera. In this case, initial data acquired by the first scan unit 100 may be 2D image data. The surface data may include surface information of the tooth 20 in the patient's oral cavity or the tooth 20 of the tooth model, and may be formed as a 3D surface model through irradiation of a structured light from an optical projector formed on the first scan unit 100. According to circumstances, the tooth 20 and the gingiva 10 having been acquired in accordance with the scan process of the first scan unit 100 may be separated and grouped into different categories, and in this case, distance measurement may not be performed with respect to the data classified into the gingiva 10 in the measurement process of the pulp cavity distance d to be described later.

Meanwhile, the first scan unit 100 may be a table scanner other than the handheld type oral scanner. The table scanner may include a tray for mounting a tooth model, and may operate to generate the surface data using the light that is reflected from the tooth model, which is put on the corresponding tray and is incident through the lens of the camera, through at least one camera formed inside the table scanner. In this case, the surface data may be scan data including surface information of the tooth model on which the patient's tooth 20 is expressed.

Further, plural pieces of surface data acquired by the first scan unit 100 may be converted into a 3D surface model through tying of the plural pieces of surface data in the unit of a group. The 3D surface model may include a point and a mesh, and may also include feature information (e.g., color information). By using the feature information, the controller may separate the tooth 20 and the gingiva from each other. Further, the 3D surface model may be composed of a voxel in the form of a pixel having a volume, and the feature information of the corresponding voxel may be included in the voxel.

Meanwhile, the pulp cavity distance measurement system according to the present disclosure may require volume data that is scan information different from the surface data acquired by the first scan unit 100. The volume data may be acquired by performing scanning of the patient's tooth as a whole, and may be used to acquire in-deep information on the patient's oral cavity. The volume data may be acquired by the second scan unit 200 formed spaced apart from the first scan unit 100. The first scan unit 100 and the second scan unit 200 may have different kinds of information intended to be acquired through the scanning.

Unlike the first scan unit 100, the second scan unit 200 may be a device that acquires the volume data by scanning the whole shape so as to penetrate the patient's tooth. The volume data acquired from the second scan unit 200 may be implemented as the 3D volume model by the controller 300. The second scan unit 200 may be, for example, at least one of a computed tomography (CT) type imaging device, an X-ray device irradiating X-rays, and a magnetic resonance imaging device acquiring a biometric tomography by using a magnetic field. In this case, the acquired volume data may be scan data in which the internal cross-sectional shape is visually processed through projecting and imaging of the oral cavity by using X-rays or ultrasonic waves. On the volume data, the part of the pulp cavity 12 may appear, and a boundary line corresponding to the pulp cavity surface 14 can also be identified. The 3D volume data being implemented from the volume data may include data from the tooth surface (enamel surface) to the pulp cavity surface 14 inside the tooth.

As described above, the first scan unit 100 can scan the surface of the tooth 20, but it is difficult to identify the internal structure of the tooth, and distance information up to the pulp cavity (e.g., pulp cavity distance) is unable to be calculated only by the tooth surface information. Meanwhile, the second scan unit 200 may identify whether there is a space inside the tooth by using the computed tomography technology, but it is difficult to visually recognize a part on which the tooth preparation can be performed due to the characteristic of the scan method for penetrating the inside of the tooth. Accordingly, the pulp cavity distance measurement system according to an embodiment of the present disclosure may utilize both the surface data acquired by the first scan unit 100 and the volume data acquired by the second scan unit 200 so as to have the advantages of the first scan unit 100 and the second scan unit 200 that acquire the scan data having different kinds of scan information. More specifically, according to the pulp cavity distance measurement system according to the present disclosure, it is possible to visually and accurately recognize the information on the tooth surface, and to mutually complement the surface data and the volume data by merging the information that can be obtained through penetration of the inside of the tooth, such as the distance information up to the pulp cavity.

Meanwhile, the first scan unit 100 and/or the second scan unit 200 may be included in the pulp cavity distance measurement system according to the present disclosure, but may be separately configured. The pulp cavity distance measurement system according to the present disclosure may receive and use the data acquired from the external first scan unit 100 and/or second scan unit 200.

The surface data and the volume data may be transmitted to the controller 300 connected to the respective scan units (first scan unit 100 and second scan unit 200) in a telecommunication manner. More specifically, the surface data acquired from the first scan unit 100 and the volume data acquired from the second scan unit 200 may be transferred to and stored in the database unit 310 formed inside the controller 300. Meanwhile, the controller 300 may correspond to a computer having a built-in microprocessor that can perform a digital calculation process, but is not limited thereto, and any configuration that can perform the data operation and processing is possible as the controller 300. The first scan unit 100 and the controller 300, and the second scan unit 200 and the controller 300 may be connected to each other by wire/wirelessly to transmit and receive data to and from each other, and in case that they are connected by wire, the data transmission/reception becomes possible through a data transmission line, whereas in case that they are connected wirelessly, the data transmission/reception becomes possible through various communication systems (Wi-Fi, Bluetooth, and Zigbee).

The controller 300 may include a data merging unit 320 that merges the transmitted surface data and volume data into one integrated data. The surface data acquired from the first scan unit 100 and the volume data acquired from the second scan unit 200 may have different file formats or different scan magnifications. Further, the surface data and/or the volume data may be 2D image data. Accordingly, in case that the surface data is the 2D data, a process of converting the data acquired as the 2D data into the 3D surface model may be performed by a processor built in the first scan unit 100, or may be performed by a calculation operation of the controller 300. Further, in case that the volume data is the 2D data, a process of converting the data acquired as the 2D data into the 3D volume model may be performed by a processor built in the second scan unit 200, or may be performed by a calculation operation of the controller 300.

The data merging unit 320 may perform alignment by adjusting the file format and/or magnification so that any one data of the 3D surface model and the 3D volume model can be mounted on other data. The alignment criterion of the 3D surface model and the 3D volume model may be the data corresponding to the tooth surface of respective pieces of data. In an embodiment, the data merging unit 320 may derive feature information of the 3D surface model and feature information of the 3D volume model, and may align the 3D surface model and the 3D volume model based on the feature information of the tooth surface. Exemplarily, the feature information may be curvature information of surface irregularities, but is not limited thereto. As the alignment method of the 3D surface model and the 3D volume model, the iterative closest points (ICP) technique, AI technology, and manual alignment may be used, but the alignment method is not limited thereto.

As described above, if the plural pieces of data are adjusted, aligned, and merged into one integrated data, the integrated data may also include data of specifications of detailed patient's oral cavity that penetrates the interior of the tooth while the integrated data has the surface shape inside the patient's oral cavity.

Further, the controller 300 may include the calculation unit 330 that calculated a specific distance in accordance with the integrated data merged by the data merging unit 320. Exemplarily, the “distance” may be calculated after the 3D surface model implemented by the surface data and the 3D volume model implemented by the volume data are aligned. In this case, the “distance” may be acquired by measuring corresponding parts of the 3D surface model and the 3D volume model. More specifically, the calculation unit 330 may calculate the distance d from the enamel surface 24 of the tooth of the 3D surface model to the pulp cavity surface 14 of the 3D volume model. The calculation unit 330 may calculate the measured distance based on the volume data acquired by the scanning of the second scan unit 200, but in case that the magnification thereof adjusted by the data integration, the calculation unit 330 may also calculate the distance by the adjusted magnification. In calculating the pulp cavity distance d, it is not necessary that the distance completely coincides with the actual distance, but it may be possible that the calculated distance has the uniform magnification so that the calculated distance corresponds to the actual distance.

Meanwhile, the pulp cavity distance d may mean the shortest distance among distances for reaching the pulp cavity surface 14 based on one point (measurement point) on the enamel surface 24. Since the pulp cavity distance d from the enamel surface 24 to the pulp cavity surface 14 appears as the shortest distance to the pulp cavity surface 14 based on the enamel surface 24, the distance to the pulp cavity surface 14, which is closest to a specific point of the enamel surface, may appear, and the therapist having recognized such distance information may proceed with the preparation by avoiding the corresponding point in the tooth preparation process, or may pay more attention during the tooth preparation. According to circumstances, on a virtual plane that is tangent to the specific point of the enamel surface to be measured, a distance to the pulp cavity surface 14, which touches the normal line of the plane that passes through the corresponding point may be used as the pulp cavity distance d.

Further, the controller 300 may further include a distance stage display unit 400 which separates the pulp cavity distance d calculated by the calculation unit 330 by patterns in accordance with a predetermined criterion, and visually displays the calculated pulp cavity distance d (e.g., the result of calculation). The distance stage display unit 400 may be a display device that can display the integrated data and information on the pulp cavity distance d included in the integrated data, but is not limited thereto. For effective display of the information on the pulp cavity distance d, the distance stage display unit 400 may separate the pulp cavity distance d into a plurality of patterns and may visually display the plurality of patterns in accordance with the size of the pulp cavity distance d.

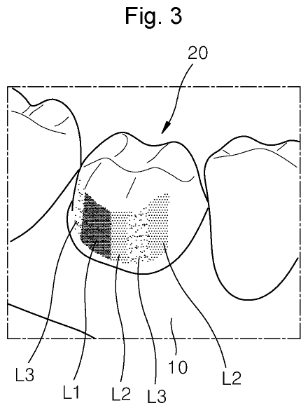

FIG. 3 is a diagram schematically illustrating information inside a tooth that is displayed by using a pulp cavity distance measurement system according to the present disclosure.

Referring to FIG. 3, merged data is displayed, in which the 3D surface model implemented from the surface data for the tooth 20 and the 3D volume model implemented from the volume data are merged each other, and the pulp cavity distance d from the enamel surface 24 to the pulp cavity surface 14 is visually displayed in the form of a plurality of different patterns, such as a first pattern L1, a second pattern L2, and a third pattern L3. In this case, if the pulp cavity distance d is 0<d<d1 according to the predetermined criterion, the first pattern L1 is given, if the pulp cavity distance d is d1≤d≤d2, the second pattern L2 is given, and if the pulp cavity distance d is d2≤d<d3, the third pattern L3 is given, and the first to third patterns L1, L2, and L3 may be displayed on the distance stage display unit 400. In this case, d1, d2, and d3 may be threshold values designated by a user, or may be threshold values corresponding to the distances in which the patient may feel discomfort in case that an additional preparation of the corresponding part is performed in the system. Exemplarily, it is described that the pulp cavity distance d is displayed with three patterns L1, L2, and L3, but is not limited thereto, and n patterns L1 to Ln may be given and displayed for predetermined sections of the pulp cavity distance d.

Further, the above-described plurality of patterns may be separated and displayed with different colors. That is, the patterns may be displayed on the distance stage display unit 400 in a manner that in case that the pulp cavity distance d is 0<d<d1 according to the predetermined criterion, the corresponding point is displayed as red, in case of d1≤d<d2, the corresponding point is displayed as blue, and in case of d2≤d<d3, the corresponding point is displayed as green. Exemplarily, three kinds of colors (red, blue, and green) have been described, but are not limited thereto, and the pulp cavity distance d may be visually displayed by using n colors. Further, as for the color depth, it is also possible to display the patterns in the form of gradation that is naturally changed in stage.

As described above, since the pulp cavity distance d is visually displayed, the therapist can visually and quickly grasp the state of the patient's tooth 20 to be treated, and can minimize the patient's discomfort by refraining from the tooth preparation in a part in which the pulp cavity distance d is short when performing the treatment, such as the tooth preparation.

Meanwhile, the pulp cavity distance d is displayed with specific patterns or colors, and in case that the enamel point is indicated by an input device (e.g., mouse cursor), the pulp cavity distance d up to the pulp cavity surface 14 of the corresponding point may be able to be displayed together. As described above, since the pulp cavity distance d can be displayed graphically and numerically, there is an advantage that the therapist can obtain desired information more accurately.

Hereinafter, a pulp cavity distance measurement method according to the present disclosure will be described. The contents that overlap the contents of the above-described pulp cavity distance measurement device will be simply mentioned or will be omitted.

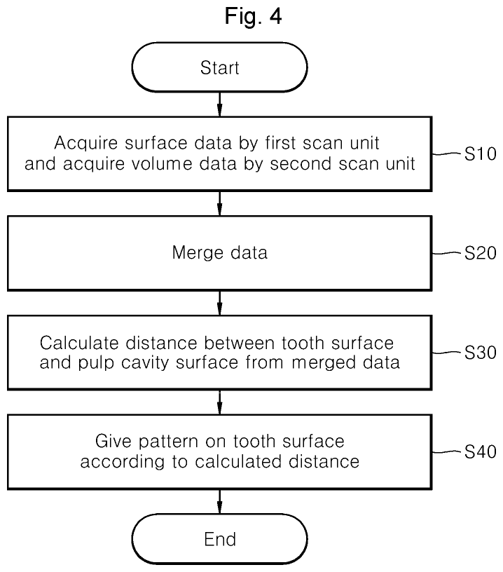

FIG. 4 is a schematic flowchart illustrating a pulp cavity distance measurement method according to the present disclosure.

Referring to FIG. 4, the pulp cavity distance measurement method according to the present disclosure may include a data acquisition step, in which the first scan unit 100 acquires surface data including surface information of a tooth or a tooth model by scanning the tooth or the tooth model, and the second scan unit 200 acquires volume data having scan information different from the scan information of the surface data by scanning the tooth. The first scan unit 100 that acquires the surface data may be a handheld type oral scanner, and the oral scanner type first scan unit 100, which has a narrow scan viewing angle, may acquire the surface data including surface information by scanning relatively small parts in overlapping manner. Alternatively, the first scan unit 100 may be a table scanner which operates to acquire the surface data including the surface information by scanning the tooth model on the whole through cameras formed around a tray formed inside the first scan unit 100. In this case, the surface data may be 2D image data, and may finally be generated as one 3D surface model through performing of an alignment process among the acquired data. The alignment process may be performed by using any algorithm that can connect the data with each other, and as an example, the alignment process may be performed by using an iterative closest point (ICP) algorithm.

Meanwhile, the surface data acquired by the first scan unit 100 may include surface information of the tooth inside the patient's oral cavity or the tooth model, and in this case, the acquired surface data may be scan information including information, such as unevenness or roughness of the patient's tooth 20. For example, the tooth 200 may be photographed by the camera included in the first scan unit 100, and the photographed patient's tooth can be generated as a digital image through the imaging sensor connected to the camera. Further, the surface data may be generated as the 2D image data, and then through the 3D conversion process, it may be converted into the 3D surface model in the above-described voxel form by the pulp cavity distance measurement system according to the present disclosure. Meanwhile, in case that the surface data is acquired through scanning of the tooth model other than the actual oral cavity of the patient, the surface data may be scan data including surface information of the model on which the patient's tooth 20 is expressed.

In the data acquisition step (S10), the volume data acquired by the second scan unit 200 may be deep data including the internal structure of the tooth 20. In this case, the volume data may have different scan information that is different from the scan information of the surface data. More specifically, the volume data may have the scan information in which the shape of the internal cross section is visually processed through the computed tomography (CT) using X-rays or ultrasonic waves and through photographing (scanning) to penetrate the tooth. However, the volume data may be acquired by a method capable of scanning the whole shape through penetration of the patient's tooth, and in this case, a magnetic resonance imaging method, which acquires a biometric tomography by using a magnetic field may be used in addition to the above-described computed tomography (CT) method and the X-ray method.

Meanwhile, if acquisition of the surface data and the volume data from the data acquisition step (S10) is completed, the data merging step (S20) may be performed by merging unit 320 of the controller 300 to merge the two acquired data (surface data and volume data) into one integrated data. The data alignment may be performed through one file format and the same magnification against the different file formats and magnifications of the surface data and the volume data. In this case, the merging may be performed so that the 3D volume model implemented from the volume data and the data for the internal specification are included onto the data generated as one 3D surface model through conversion of the surface data into 3D data.

Further, the pulp cavity distance measurement method according to the present disclosure may further include a distance calculating step (S30). In the distance calculating step (S30), after aligning the 3D surface model and the 3D volume model in the data merged in the data merging step (S20), the controller 300 (more specifically, a calculation unit 330) may acquire the distance by measuring the corresponding parts of the 3D surface model and the 3D volume model. Exemplarily, in the distance calculating step (S30), after the tooth surface of the 3D surface model and the tooth surface of the 3D volume model are aligned with each other, the distance between the corresponding points may be calculated. More specifically, in the distance calculating step (S30), it is possible to calculate the distance d from the enamel surface 24 of the tooth 20 of the 3D surface model to the pulp cavity surface 14 of the 3D volume model. In this case, the alignment criterion of the 3D surface model and the 3D volume model may be data corresponding to the tooth surface in respective pieces of data. The pulp cavity distance d is not necessarily required to be expressed as the distance from the enamel surface of the patient's tooth to the pulp cavity surface 14, and may be expressed to form a proportional relationship by applying a specific multiple of the measured distance. In this case, the pulp cavity distance d may be the shortest distance among distances for reaching the pulp cavity surface 14 based on one point (measurement point) on a specific enamel surface. According to circumstances, the pulp cavity distance d may be the distance measured along the normal direction that is vertical to the tangent line of the measurement point.

Meanwhile, the pulp cavity distance d acquired by the distance calculating step (S30) may be given in the form of a pattern by the controller 300 in order to visually display the pulp cavity distance on the display device such as the distance stage display unit 400 (pattern giving step (S40)). In this case, the given patterns are as described above in the pulp cavity distance measurement system according to the present disclosure, and the pulp cavity distance d may be divided by sections, and the divided plurality of patterns or colors may be given to the enamel surface.

Further, in case that the therapist indicates the specific point of the enamel surface where the pulp cavity distance d is intended to be grasped with a cursor simultaneously with displaying of the pulp cavity distance d graphically (pattern or color, or combination of pattern and color), the information on the pulp cavity distance d can be numerically displayed, and thus the therapist can be helped in the treatment process of the patient.

The above explanation of the present disclosure is merely for exemplary explanation of the technical idea of the present disclosure, and various changes and modifications may be possible in a range that does not deviate from the essential characteristics of the present disclosure by those of ordinary skill in the art to which the present disclosure pertains.

Accordingly, embodiments disclosed in the present disclosure are not to limit the technical idea of the present disclosure, but to explain the technical idea, and the scope of the technical idea of the present disclosure is not limited by such embodiments. The scope of the present disclosure should be interpreted by the appended claims, and all technical ideas in the equivalent range should be interpreted as being included in the scope of the present disclosure.

### INDUSTRIAL APPLICABILITY

The present disclosure provides a pulp cavity distance measurement system and method, which can minimize patient's discomfort during the tooth preparation by aligning the 3D surface model and the 3D volume model and then calculating and displaying the distance between them.

1. A pulp cavity distance measurement system comprising:
a database unit configured to acquire surface data of a tooth or a tooth model and volume data of the tooth; and
a calculation unit configured to calculate a distance between corresponding parts of a 3D surface model implemented from the surface data and a 3D volume model implemented from the volume data after aligning the 3D surface model and the 3D volume model.

2. The system of claim 1, further comprising a first scan unit configured to acquire and transfer the surface data to the database unit.

3. The system of claim 1, further comprising a second scan unit configured to acquire and transfer the volume data to the database unit.

4. The system of claim 1, wherein the 3D volume model comprises data from a surface of the tooth to a pulp cavity surface inside the tooth.

5. The system of claim 1, wherein the calculation unit is configured to calculate a distance after aligning between a tooth surface of the 3D surface model and a tooth surface of the 3D volume model.

6. The system of claim 1, wherein the calculation unit is configured to calculate a distance from a tooth surface of the 3D surface model to a pulp cavity surface of the 3D volume model.

7. The system of claim 6, wherein the distance is the shortest distance from a measurement point of the 3D surface model to the pulp cavity surface of the 3D volume model.

8. The system of claim 1, further comprising a distance stage display unit configured to visually display the distance.

9. The system of claim 8, wherein the distance stage display unit is configured to separately display a plurality of patterns in accordance with a size of the distance.

10. The system of claim 9, wherein the plurality of patterns are separately displayed with different colors.

11. A pulp cavity distance measurement method comprising:
a data acquisition step of acquiring surface data of a tooth or a tooth model and volume data of the tooth;
a data merging step of merging a 3D surface model implemented from the surface data with a 3D volume model implemented from the volume data; and
a distance calculating step of calculating a distance between corresponding parts of the 3D surface model and the 3D volume model after aligning the 3D surface model and the 3D volume model.

12. The method of claim 11, wherein the distance calculating step calculates a distance after aligning between a tooth surface of the 3D surface model and a tooth surface of the 3D volume model.

13. The method of claim 11, wherein the distance calculating step calculates a distance from a tooth surface of the 3D surface model to a pulp cavity surface of the 3D volume model.

14. The method of claim 13, wherein the distance calculated in the distance calculating step is the shortest distance from a measurement point of the 3D surface model to the pulp cavity surface of the 3D volume model.

15. The method of claim 11, further comprising a pattern giving step of appearing in a pattern form on a distance stage display unit in order to visually display the distance calculated in the distance calculating step.

16. The method of claim 15, wherein a plurality of patterns are formed in accordance with the distance calculated in the distance calculating step, and are separately displayed with different colors.
